(19) United States
(12) Patent Application Publication
(10) Pub. No.: US 2022/0130529 A1
(43) Pub. Date: Apr. 28, 2022

(54) MEDICAL FACILITY REMOTE PATIENT ASSIGNMENT SYSTEMS AND METHODS

(71) Applicants: Qatar Foundation for Education, Science and Community Development, Doha (QA); Qatar University, Doha (QA)

(72) Inventors: Abubakr O. Al-Abbasi, Doha (QA); Lutfi Samara, Doha (QA); Ridha Hamila, Doha (QA); Naofal Al-Dhahir, Doha (QA); Saeed Salem, Doha (QA)

(21) Appl. No.: 17/510,986

(22) Filed: Oct. 26, 2021

Related U.S. Application Data
(60) Provisional application No. 63/105,636, filed on Oct. 26, 2020.

(51) Int. Cl. G16H 40/20; G06Q 10/10; G06N 3/08

(52) U.S. Cl. CPC G16H 40/20; G06N 3/08; G06Q 10/1095

(57) ABSTRACT

The provided systems and methods are designed to account for the requests of multiple patients demanding a form of medical intervention from a medical facility (MF) in their vicinity. Unlike the majority of existing approaches, the provided systems and methods differentiate between patient requests by prioritizing the requests with higher urgency over others that can tolerate more delay. Moreover, the provided systems and methods enjoy lower design complexity since the instantaneous queue length (number of patients) in each medical facility is not necessarily needed in at least some aspects. Instead, in such aspects, only average queue length is involved in taking the dispatch actions for patient requests to medical facilities. The intervention can vary from a routine consultation/meeting with a healthcare service provider to urgent hospital admission.

### PRIORITY CLAIM

The present application claims priority to and the benefit of U.S. Provisional Application 63/105,636, filed Oct. 26, 2020, the entirety of which is herein incorporated by reference.

### BACKGROUND

Typically, patient scheduling is either independent across hospitals or depends on patients' choices (e.g., calling a hospital or multiple ones to book, though the patient may or may not get a clinical bed or visit). These techniques involve human interactions, delay, and in many scenarios, are not optimal. Further, patient scheduling sometimes is based on the results of the screening and triaging process that takes place once a patient enters the treatment area. This results in long queues that prolong the stay of patients at the medical facility (MF), thus endangering other patients. In an example, the COVID-19 outbreak imposed an unprecedented pressure on healthcare systems around the world. Accordingly, a need exists for a solution to efficiently manage healthcare facilities for remote patient scheduling at medical facilities.

### SUMMARY

The present disclosure provides new and innovative systems and methods for the efficient distribution of patients across heterogeneous medical facilities. In an example, a system for remote patient assignment includes a processor in communication with a memory. The processor is configured to receive a scheduling request from a computing device of a patient over a network; receive information from each of a plurality of medical facilities; determine an estimated service time for the patient to be treated at each of the plurality of medical facilities, wherein the estimated service time includes an estimated travel time for the patient to arrive at a respective medical facility, an estimated waiting time for the patient at the respective medical facility, and an estimated consultation time with a medical professional for the patient; select a medical facility of the plurality of medical facilities that minimizes a sum of the determined estimated service time for the patient and a probability that the determined estimated service time for the patient is greater than or equal to a predefined threshold; and assign the patient to the selected medical facility.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 illustrates a block diagram of an example system for assigning patients to a medical facility for an appointment, according to an aspect of the present disclosure.

FIG. 2 illustrates a flow chart of an example method for assigning patients to a medical facility for an appointment, according to an aspect of the present disclosure.

FIG. 3 illustrates a graph showing the convergence of the provided model-based method.

FIG. 4 illustrates a graph depicting the performance of three optimization approaches.

FIG. 5 illustrates a graph showing the effect of increasing the rate of patients requests.

FIG. 6 illustrates a graph showing the behavior of weighted STTP versus the threshold.

FIG. 7 illustrates a graph depicting the EST versus the number of patients per group.

FIG. 8 illustrates a graph depicting the performance of the provided DRL-based approach when increasing the rate of patient requests.

### DETAILED DESCRIPTION

The present disclosure provides systems and methods for the efficient distribution of patients across heterogeneous medical facilities. As used herein, a medical facility (MF) may be a hospital, health center, clinic, etc. The provided systems and methods are designed to account for the requests of multiple patients demanding a form of medical intervention from a medical facility (MF) in their vicinity. Unlike the majority of existing approaches, the provided systems and methods differentiate between patient requests by prioritizing the requests with higher urgency over others that can tolerate more delay. Moreover, the provided systems and methods enjoy lower design complexity since the instantaneous queue length (number of patients) in each medical facility is not necessarily needed in at least some aspects. Instead, in such aspects, only average queue length is involved in taking the dispatch actions for patient requests to medical facilities. The intervention can vary from a routine consultation/meeting with a healthcare service provider to urgent hospital admission.

The provided system provides a framework that distributes the patients across the heterogeneous medical facilities so that a weighted sum of the expected service time and service time tail probability for all patients is minimized. The provided system prioritizes the patients with severe/critical conditions over others that can tolerate more delay. Based on the model, an optimization problem is formulated as a convex combination of both expected service time and service time tail probability metrics, and an efficient iterative algorithm is used to solve it. The inventors demonstrated that the provided system provides a performance improvement (up to 50%) as compared to other algorithms and typical solutions.

The provided method's main objective is the minimization of a weighted sum of expected service time (EST) and service time tail probability (STTP), for all patients. The provided method may utilize model-based or reinforcement learning (RL) model-free holistic optimization frameworks to optimize a convex combination of a weighted sum of EST and STTP. The model-free approach leverages RL techniques to learn the systems parameters and thus efficiently assigns patients requests to MFs such that both MF limitations and patient requirements are satisfied. The method accounts for the patients' needs and medical facilities' heterogeneities when scheduling the patients' requests. In an example, the service time of a patient consists of three components: travel time to reach the medical facility, the waiting time in the medical facility, and finally the consultation time he/she spends with the medical facility. This is a critical aspect to consider in times of the outbreak of infectious diseases, where social distancing has been recommended as a mean to minimize the disease's spread amongst the population.

The provided systems and methods can be used by healthcare providers or can be adopted by relevant public health entities to efficiently schedule patients to medical facilities. The provided systems and methods can be adopted for the general scheduling of patients to adequate medical facilities. In some examples, the provided systems and methods can be especially useful in the case of the sudden spread of a communicable disease that requires the avoidance of crowded places and social distancing. For instance, the provided systems and methods help efficiently minimize the time spent by a patient in his/her visit to the medical facility, and by the fair allocation of patients to different heterogeneous medical facilities such that overcrowding in the medical facilities can be avoided. The provided systems and methods can be used by a single private agent (e.g., insurance company) to manage its customers, or by a governmental entity (ministry of health) to manage public medical facilities. In general, the provided system can be scaled to manage the medical resources in a county-wise, city-wise, state-wise or even a country-wise fashion, depending upon the capability of a service provider.

It should be understood that while the presently disclosed method and system are described as being used only by patients and medical facilities, other parties may use the presently disclosed method and system for patient scheduling purposes. For example, in the case of patients, a family member may be authorized by the patient to use the method or system on the patient's behalf. In some instances, automatons or bots may also be used to act on behalf of a patient.

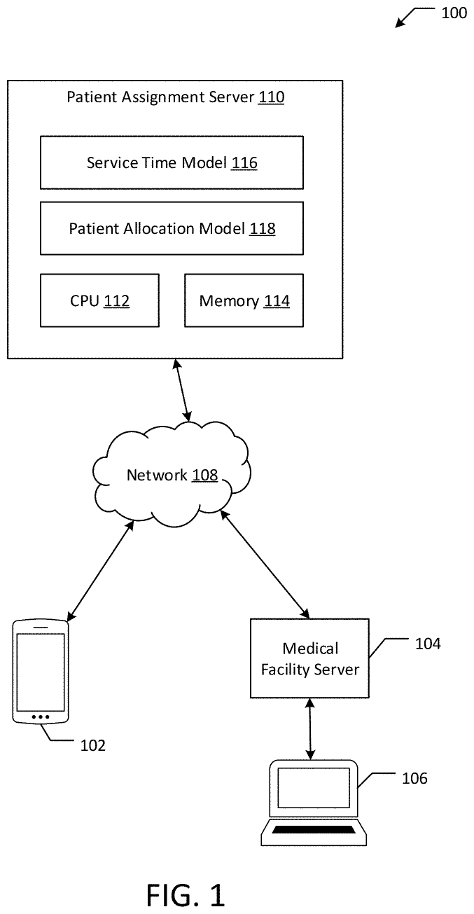

FIG. 1 illustrates an example system 100 that provides for patient scheduling. The system 100 may include a patient assignment server 110 communicatively coupled to one or more patient terminals 102 and one or more medical facility servers 104 via a network 108. The network 108 can include, for example, the Internet or some other data network, including, but not limited to, any suitable wide area network or local area network. The example patient assignment server 110 is configured to receive scheduling requests for a specific health service from patients and a patient's location via the patient terminals 102 and capacity/timing information from medical facilities via the medical facility servers 104, and assign each patient to a particular medical facility to satisfy each patient's needs while taking into account the different capabilities of each medical facility. The patient assignment server 110 is responsible for maintaining relevant information related to patient scheduling, such as updating a state of the current number of patients at each medical facility, occupancy state of each medical facility, wait times at each medical facility, etc. These states are continuously updated based on patient assignment decisions. A medical facility professional may access the patient assignment server 110 via a medical facility professional terminal 106. In other examples, the medical facility professional terminal 106 may connect directly to the network 108, bypassing the medical facility server 104.

The patient terminal 102 and/or the medical facility professional terminal 106 may include any type of device including a smartphone (e.g., the patient terminal 102 in FIG. 1), a cellular phone, a tablet computer, a laptop computer (e.g., the medical facility professional terminal 106 in FIG. 1), a workstation, smart-eyewear, smartwatch, etc. The medical facility server 104 may include any healthcare computer system and/or network, such as an enterprise system. The medical facility server 104 may maintain patient scheduling information and other facility-related scheduling information such as data on patient waiting times and consultation lengths for various medical issues.

The example patient terminal 102 may include an application (e.g., an App). The application is configured to acquire registration information, acquire scheduling requests, display available medical facilities in a patient's area, receive a patient assignment to a medical facility, and display the patient assignment. The application may operate in connection with the patient assignment server 110, which determines to which medical facility a patient should be assigned. Throughout this disclosure, various examples of the application are discussed to explain the presently disclosed method and system. It should be appreciated, however, that in various embodiments the application may be in addition to, or replaced by, a website hosted or otherwise provided by the patient assignment server 104. In such embodiments, patients and medical facility professionals may use respective terminals 102 and 106 to access the website of the patient assignment server 104. A web browser on the terminals 102 and 106 is used to access the website for acquiring registration information, acquiring scheduling requests, displaying available medical facilities in a patient's area, receiving a patient assignment to a medical facility, and displaying the patient assignment.

Also shown in FIG. 1, is an example diagram of the patient assignment server 110. The patient assignment server 110 includes different components that are representative of computational processes, routines, and/or algorithms. In some embodiments, the computational processes, routines, and/or algorithms may be specified in one or more instructions stored on a computer readable medium that, when executed by a processor of the patient assignment server 110, cause the patient assignment server 110 to perform the operations discussed below. The processor may be a CPU 112, an ASIC, or any other similar device. For example, all or part of the computational processes, routines, and/or algorithms may be implemented by the CPU 112 and the memory 114. In other embodiments, the components of the patient assignment server 110 may be combined, rearranged, removed, or provided on a separate device or server.

The holistic optimization framework of the patient assignment server 110 is designed to optimize a convex combination of a sum of expected service time and service time tail probability in order to assign a patient to a medical facility. In some aspects, the patient assignment server 110 may include at least one model trained to determine which medical facility the patient should be assigned to. Prediction models may be used by utilizing the data fed by the environment, i.e. input data from the medical facilities and the patients. The prediction models yield the parameters used in solving an optimization problem, which are the estimated time of arrival (ETA) data, the service and arrival rates at each of the involved MFs. Then, these parameters may be used in solving an optimization problem to determine the most suitable MF to a patient's request. Different objective functions can be formulated and optimized depending on the goals of the designer using any suitable approach or algorithm.

In an example, the patient assignment server 110 may include at least one service time model 116 trained to estimate an amount of time it would take for a patient to get treated at a particular medical facility given the patient's location. The at least one service time model 116 may be implemented by one or more suitable machine learning models, including one or more supervised learning models, unsupervised learning models, or other types of machine learning models. For example, the at least one service time model 116 may be implemented as one or more of a neural network (e.g., neural networks with dense mapping, convolutional neural networks, or recurrent neural networks), a decision tree model, a support vector machine, and a Bayesian network.

In another example, the patient assignment server 110 may include at least one patient allocation model 118 trained to assign a patient to a particular medical facility based on the service times estimated by the service time model 116 for various medical facilities. The at least one patient allocation model 118 may be implemented by one or more suitable machine learning models, including one or more supervised learning models, unsupervised learning models, or other types of machine learning models. For example, the at least one patient allocation model 118 may be implemented as one or more of a neural network (e.g., neural networks with dense mapping, convolutional neural networks, or recurrent neural networks), a decision tree model, a support vector machine, and a Bayesian network.

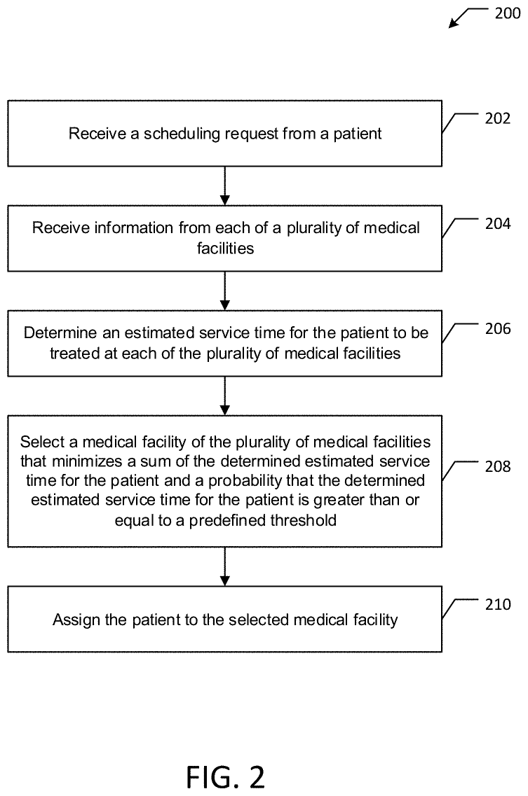

FIG. 2 illustrates a flow chart of an example method 200 for assigning patients to medical facilities in response to scheduling requests from those patients. Although the example method 200 is described with reference to the flowchart illustrated in FIG. 2, it will be appreciated that many other methods of performing the acts associated with the method 200 may be used. For example, the order of some of the blocks may be changed, certain blocks may be combined with other blocks, and some of the blocks described are optional. The method 200 may be performed by processing logic that may comprise hardware (circuitry, dedicated logic, etc.), software, or a combination of both.

The example method 200 may begin by receiving a scheduling request from a patient (block 202). For example, a patient may submit a scheduling request for a particular medical service using an application on the patient terminal 102. The patient assignment server 110 may receive this scheduling request. In various aspects, a scheduling request may include patient information (name, age, gender, etc.), a description of the medical issue for which treatment is sought, the patient's location, a distance radius within which the patient would like to be treated, or other suitable information for scheduling a patient for a medical visit.

Information may also be received from multiple medical facilities (block 204). For example, the patient assignment server 110 may receive data from a medical facility server 104 of each medical facility participating in the system 100. The data from a medical facility server 104 may include a current quantity of patients at the medical facility, an occupancy state of the medical facility, an availability state of the medical facility, an average wait time at the medical facility, or other suitable information related to a capacity of the medical facility or an amount of time it would take for a patient to get treated at the medical facility. The states of each medical facility may be continuously updated based on the patient assignment decisions.

An estimated service time for the patient to be treated at each of the medical facilities may then be determined (block 206). As used herein, a service time is defined as the sum of the travel time to reach the MF, the waiting time, and consultation time. Throughout this description, visit time, seen time, and consultation time may be used interchangeably. In some aspects, the patient assignment server 110 may use at least one model (e.g., the service time model 116 and/or the patient allocation model 116) to determine the estimated service time at each medical facility. An example model-based approach will now be described for such aspects.

It can be assumed that there are P potential patients requesting a specific health service from a MF (e.g., hospital, health center, clinic etc). It can be assumed that every patient requests a medical service through a mobile application asynchronously. Moreover, it can be denoted by p the p-th patient, i.e., pϵI={1, . . . , P}. Furthermore, it can be assumed that patient p generates requests at a rate of λₚ. The rates of requests are assumed to follow a Poisson process and are independent across different patients. Hence, for every patient p, the inter-request time is exponentially distributed with rate λₚ.

The total service time of a request made by patient p consists of three components: (i) travel time to reach the MF h, (ii) waiting for service at the MF h, and (iii) consultation/seen time by the doctor. For travel time, Let t_{p,h} denote the random travel time for patient p to reach MF h. This time is determined by the distance D_{p,h} between patient p and MF h, as well as the speed C m/s of the vehicle used to reach the MF h, e.g., t_{p,h}=D_{p,h}/C. In addition, to capture the traffic uncertainty and variability, a random variable t_{p,h} with mean α_{p,h}, for patient p heading to MF h may be added to the fixed term D_{p,h}/C. As it can be commonly assumed, τ_{p,h} follows a normal distribution with mean ∝_{p,h} and variance σₚ²_{p,h} and thus τ_{p,h} accounts for the randomness of the travel time. Hence, the average travel time for patient p to reach MF h is given by Equation 1 below.

$$𝔼 [ {t}_{p , h} ] = \frac{{D}_{p , h}}{C} + {∝}_{p , h} \qquad ( 1 )$$

In Equation 1, [symbol] [t_{p,h]} is the estimated travel time for the patient, p is the patient, h is the respective medical facility, t_{p,h} is a random travel time for the patient to reach the respective medical facility, D_{p,h} is a distance between the patient and the respective medical facility, C is a speed of travel, and ∝_{p,h} is a mean value of a random variable accounting for the randomness of the travel time.

In some aspects, to estimate travel time for the patient, the service time model 116 may include a fully connected multi-layer perception network (e.g., neural network). The network may consist of two hidden layers with width of 64 units and rectifier non-linearity in one example. A latitude, longitude positional pair may be an input to this learning model to estimate a patient's travel time. The output of this model is the travel time of patient p to the MF h. The output of this neural network is built to give the expected travel time between the patient and the MF.

In at least some aspects, a waiting time for service at the MF may be an average waiting time for service at the MF. In other aspects, the waiting time for service at the MF may be calculated in other suitable ways.

Turning to consultation time, for a patient p assigned to MF h for service, it can be assumed that the consultation/seen time follows a shifted exponential distribution ƒ_{p,h}(s) given by Equation 2 below. In Equation 2, μ_{p,h}=μₕ/Vₚ, β_{p,h}=βₕVₚ, p is the patient, h is the respective medical facility, βₕ is a minimum consultation time at the respective medical facility, μₕ is an average consultation time at the respective medical facility, Vₚ is a total time of a visit by a patient at the respective medical facility, and s is a type of service for the consultation.

$${f}_{p , h} ( s ) \{ \begin{matrix}{μ}_{p , h} {e}^{- {μ}_{p , h} ( s - {β}_{p , h} )} & s \geq {β}_{p , h} \\ 0 & s < {β}_{p , h}\end{matrix} \qquad ( 2 )$$

The value of μ_{p,h} decreases in proportion to Vₚ while β_{p,h} increases in the exponential consultation time distribution. Thus, the visit time depends on the type of consultation and, hence, it scales differently from one patient to another according to the type of visit. It is worth noting that the shift part (β_{p,h}) represents the minimum of seen time of patient p to be successfully checked by the doctor. Furthermore, the exponential part (1/,μ_{p,h}) accounts for any randomness that renders the consultation time non-deterministic. Unlike the exponential service model, the adopted shifted exponential model provides flexibility for a more realistic modeling of the health service. Moreover, the exponential distribution is a special case with β_{p,h}=0. Similarly, a deterministic service time can follow as a special case by making the exponential rate very high. A shifted two-parameter may be chosen in order to simulate general distributions with parameters (β_{p,h}, μ_{p,h}). When the shift parameter is much larger than the random part of the service time (1/μ_{p,h}), it can approximate the deterministic models. In contrast, when the shift parameter is much smaller than 1/,μ_{p,h}, it approximates the exponential distribution. In the general case where no parameter is dominating, the service time includes the two components: fixed time and a random time. Hence, the shifted exponential distribution includes the exponential and deterministic/general distributions as special cases. Let M_{p,h}(τₕ)=Ε[e^{τ}^{ₕ}^{S}^{_{p,h}}] be the moment generating function (MGF) of the consultation time of a patient p at MF h, S_{p,h}. Then, M_{p,h}(τₕ) is given according to Equation 3 below. The MGF of the travel time distribution t_{p,h} can be defined in a similar fashion.

$${M}_{p , h} ( {τ}_{h} ) = \frac{{μ}_{p , h}}{{μ}_{p , h} - {τ}_{h}} {e}^{{β}_{p , h} {τ}_{h}} \qquad ( 3 )$$

In some aspects, the patient assignment server 110 may use reinforcement learning to determine the estimated service time at each medical facility.

A medical facility is then selected for the patient out of the multiple medical facilities (block 208). In at least some aspects, the objective is to dispatch the requests of patients to MFs in such a way that a sum of the total service time and its tail probability is minimized. The sum may be weighted. It may be assumed that each visit request generated by patient p needs a total of Vₚ units of time. The request can be assigned to any MF h, hϵ{1, 2, . . . , H} for service. Further, the service can be assumed to be non-preemptive so patients cannot be interrupted if they are already being served.

In some aspects, the following scheduling approach for patient requests may be used that includes parameters that can be used to optimize the total service time. Upon the arrival of a patient request, one of the MFs is selected to serve it. The optimal scheduling strategy has to consider many factors including the travel time to reach the MF, Q state (number of patients) for each center, the patient's condition, and all patients who are not fully served. The scheduling approach may consider all different control parameters, i.e., the scheduling decisions. To provide differentiated service levels, requests of patient p is assigned to the queue of MF hϵH, with probability q_{p,h}≥0. Note that q_{p,h} is the probability of serving a request of patient p from MF h. Each MF h maintains its own queue and the patients in each queue are served under a First Come First Serve policy (FCFS). Although FCFS is adopted in this example, the provided solution can host various queueing approaches in other examples.

Having defined the prioritized scheduling policy, EST and STTP may be defined. Let L_{p,h} denote denote the random variable corresponding to the total service time that patient spends if assigned to MF. Recall that L_{p,h} depends on three components, (i) t_{p.h}—travel time needed by patient to reach the MF, (ii) Qₕ—waiting time in the queue at the MF, and (iii) S_{p,h}—consultation time for a patient at MF. The STTP of a request for patient p is defined as the probability that the total service time of patient p is greater than or equal to a predefined threshold δₚ, for a given δₚ. In order to serve a patient, one MF may first be selected to serve its request. MF is chosen with probability q_{p,h} to serve patient's request. Since the key bottleneck is the limited number of MFs, service requests have to wait in the queue. Thus, if the MFs are occupied while serving other patients, the incoming new visit requests have to wait before being served. Under prioritized scheduling, the arrival of requests at MF follows a Poisson process with a rate Λₕ according to Equation 4 below.

$${Λ}_{h} = {\sum }_{p = 1}^{P} {q}_{p , h} {λ}_{p} \qquad ( 4 )$$

The consultation time of a request for a patient p if served through MF h, denoted by S_{p,h}, is given by (β_{p,h}+1/μ_{p,h}). Hence, the consultation/visit time at center h is given according to Equation 5 below. The MGF for the consultation time of Sₕ is given according to Equation 6 below.

$$\begin{matrix}{S}_{h} = {S}_{p , h} with probability \frac{{p}_{p , h} {λ}_{p}}{{Λ}_{h}} ∀ h . & ( 5 ) \\ {M}_{h} ( τ ) = {\sum }_{p = 1}^{P} \frac{{q}_{p , h} {λ}_{p}}{{Λ}_{h}} ( \frac{{μ}_{p , h} {e}^{{β}_{p , h} τ}}{{μ}_{p , h} - τ} ) , \\ for any τ > 0 , and τ < {μ}_{p , h} . & ( 6 )\end{matrix}$$

In at least some aspects, having characterized the consultation time distribution, the average service time and MGF of the STTP, L_{p,h} can be characterized using the Pollaczek-Khinchine (PK) formula for M/G/1 queues, since the request pattern is Poisson and the service time is generally distributed. Then, for a given request for patient p, the EST may be given by Equation 7 below. In Equation 7,

$$𝔼 [ {S}_{h} ] = {\sum }_{p = 1}^{P} \frac{{q}_{p , h} {λ}_{p}}{{Λ}_{h}} ( 𝔼 [ {S}_{p , h} ] ) , 𝔼 [ {S}_{p , h} ] = {β}_{p , h} + \frac{1}{{μ}_{p , h}} ,$$

Λₕ is an arrival rate of requests at the respective medical facility, Lₚ is a service time of the patient, μ_{p,h}=μₕ/Vₚ, β_{p,h}=βₕVₚ, p is the patient, h is the respective medical facility, βₕ is a minimum consultation time at the respective medical facility, μₕ is an average consultation time at the respective medical facility, Vₚ is a total time of a visit by a patient at the respective medical facility, q_{p,h} is a probability of serving a request of a patient from the respective medical facility, D_{p,h} is a distance between the patient and the respective medical facility, C is a speed of travel, and ∝_{p,h} is a mean value of a random variable accounting for the randomness of the travel time.

$$𝔼 [ {L}_{p} ] = {\sum }_{h = 1}^{H} {q}_{p , h} [ ( \frac{{D}_{p , h}}{C} + {∝}_{p , h} ) + \frac{{Λ}_{h} 𝔼 [ {S}_{h}^{2} ]}{2 ( 1 - {Λ}_{h} 𝔼 [ {S}_{h} ] )} + 𝔼 [ {S}_{p , h} ] ] \qquad ( 7 )$$

Further, the STTP may be given by Equation 8 below. In Equation 8,

$$0 < {τ}_{h} < {μ}_{p , h} , {ρ}_{h} = {Λ}_{h} 𝔼 [ {S}_{h} ] , \\ 𝔼 [ {S}_{h} ] = {\sum }_{p = 1}^{P} \frac{{q}_{p , h} {λ}_{p}}{{Λ}_{h}} ( 𝔼 [ {S}_{p , h} ] ) , 𝔼 [ {S}_{p , h} ] = {β}_{p , h} + \frac{1}{{μ}_{p , h}} ,$$

Lₚ is a service time of the patient, Λₕ is an arrival rate of requests at the respective medical facility, μ_{p,h}=μₕ/Vₚ, β_{p,h}=βₕVₚ, p is the patient, h is the respective medical facility, βₕ is a minimum consultation time at the respective medical facility, μₕ is an average consultation time at the respective medical facility, Vₚ is a total time of a visit by a patient at the respective medical facility, q_{p,h} is a probability of serving a request of a patient from the respective medical facility, and δₚ is the predefined threshold.

$$ℙ ( {L}_{p} \geq {δ}_{p} ) \leq {\sum }_{h = 1}^{H} \frac{{q}_{p , h} ( 1 - {ρ}_{h} ) {τ}_{h} {e}^{- {τ}_{h} {δ}_{p}}}{{τ}_{h} - {Λ}_{h} ( {M}_{h} ( {τ}_{h} ) - 1 )} \times ( {e}^{{D}_{p , h} {τ}_{h} / C} {e}^{{σ}_{p , h}^{2} {τ}_{h}^{2} / 2} ) ( \frac{{μ}_{p , h} {e}^{{β}_{p , h} {τ}_{h}}}{{α}_{p , h} - {τ}_{h}} ) \qquad ( 8 )$$

A joint EST and STTP optimization problem for multiple patients with heterogeneous characteristics of medical facilities may be used. In at least some aspects, and aim may be to minimize a weighted sum up the EST and STTP over the choice of prioritized decisions q and the auxiliary variable τ. Since this is a multi-objective optimization, the objective can be modeled as a convex combination of the two metrics. Let [symbol] ₚ be a trade-off factor, for patient p, that determines the relative significance of STTP in the optimization problem, where [symbol] ₚϵ[0,1]. Further, let ωₚ denote the severity of a patient's health condition. This weighting may provide more flexibility in scheduling patient requests, where patients with higher weights (e.g., more time sensitive) can be prioritized over patients who are less sensitive to delays. The EST averaged over all patient requests may be minimized. Then, the STTP of all patients, averaged overall patient requests, may be minimized. As such, optimizing a combination of the two adopted metrics while using trade-off factor [symbol] ₚ is formulated according to Equation 9 below. In Equation 9, ωₚ is a level of severity of a health condition of the patient, [symbol] ₚ is a trade-off factor, for the patient, that determines the relative significance of STTP, Lₚ is a service time of the patient, δₚ is the predefined threshold, and [symbol] (Lₚ≥δₚ) is the STTP.

$${min}_{q , τ} {\sum }_{p = 1}^{P} {ω}_{p} [ ( 1 - {ϛ}_{p} ) 𝔼 [ {L}_{p} ] + {ς}_{p} ℙ ( {L}_{p} \geq {δ}_{p} ) ] \\ {p}_{h} < 1 , ∀ h \\ {Λ}_{h} ( {M}_{h} ( {τ}_{h} ) - 1 ) < {τ}_{h} , ∀ h \\ {q}_{p , h} \geq 0 , ∀ p , h \\ 0 < {τ}_{h} < {μ}_{p , h} , ∀ p , h . \qquad ( 9 )$$

Varying [symbol] ₚ=1 to [symbol] ₚ=0 in Equation 9 results in a solution that spans the possible solutions that minimize the STTP to the ones that minimize the EST of the patient. The constraints in Equation 9 ensure that the load intensity at MF is less than one for a stable system, that the MGF of the service time exists, and feasibility of the decision variables. The optimization over q can offer significant flexibility over queue-based policies such as choosing the shortest-queue MF for scheduling the patients. This is because queue-based scheduling does not differentiate between patient requests based on their weights/severity. Unlike these typical approaches, the presently disclosed method 200 prioritizes requests according to their weights so that requests with higher weights are prioritized more to further reduce the EST and STTP and thus can help improve the efficiency of the overall healthcare system.

In some aspects, an efficient algorithmic solution may be used for solving the joint optimization problem. To develop an algorithmic solution, since the problem is non-convex, an alternating optimization algorithm may be used for the problem where only one variable is optimized at a time (while other variables are fixed). The joint EST and STTP given in Equation 9 is optimized over two sets of variables: prioritized scheduling probabilities q, and auxiliary variables τ. The provided algorithm divides the problem into two subproblems that optimize one variable while fixing the other. The two sub-problems are labeled as (i) Scheduling q-Optimization which optimizes q for a given τ, (ii) τ-Optimization which optimizes τ for a given q. The algorithm is summarized as follows. First, q and τ may be initialized in the feasible set. Then, q-Optimization is run using current values of τ to get new values of q. Then, τ-Optimization is run using current values of q to get new values of τ. In other aspects, τ-Optimization may be run prior to q-Optimization. In can be easily shown that τ-Optimization is convex since the objective function and the constraints are convex with respect to each variable individually. Since q-Optimization is a non-convex optimization problem, a Successive Upper-Bound Minimization (SUM) algorithm may be used to solve this sup-problem.

In some aspects, since arrival rate and service time may be time dependent, two online algorithms for minimizing the weighted objective may be used. The online algorithms may account for the heterogeneity among patients and MFs. The first online algorithm (e.g., Randomized Prioritized Scheduling (RPS)) is developed based on the stationary scheduling decisions which result from the solution of the optimization problem defined in Equation 9. The second algorithm is a pull-based algorithm (PBA).

For the RPS algorithm, the rates of patients requests λₚ can be estimated using a window-based method. Under this setting, a window-size of ΔW is chosen, and the decisions in a window are based on the estimated arrival requests rates from the preceding window. Using these estimated arrival rates, the solution for the optimization problem in Equation 9 gives the optimal offline scheduling decisions, q. According to these stationary scheduling probabilities, a randomized online policy can be obtained.

For the PBA algorithm, patients' requests are stored in a local queue (e.g., at the patient assignment server 110). The requests are sorted based on their weights ωₚ, where higher ωₚ's are placed at the head of the queue. Then, requests are processed according to their priority levels. The PBA algorithm pulls the status of the MFs and sends the head of the queue request to the MF with the least estimated load left.

In some instances, the arrival rates of the patients requests and the mean service times of the MFs can be difficult to estimate in practice. For example, the patient generally has no prior knowledge of the waiting in each MF. Further, it is a difficult problem, for the service provider to accurately estimate the service times and arrival rates, especially under outbreak circumstances and emergencies. In addition, the patient has no knowledge about the MFs' occupancy. In such instances, an example model-free approach that leverages reinforcement learning (RL) may be used which will now be described.

While various queuing-based models have been adopted in the healthcare services, many of the assumptions such as the arrival and service rates are difficult to obtain. It is then essential to solve the allocation problem while adopting the least assumptions. RL is an efficient tool that can be used effectively to solve the scheduling of patients in a model-free scenario. The adopted RL algorithm is model-free, i.e. it does not require any a-priori knowledge of the nature of the used data. Since arrival and service rates of patients can be time variant, a model-free approach can be leveraged to efficiently assign patients to MFs.In each step, the RL algorithm takes a suitable action to maximize the reward. Unlike other machine learning algorithms where ground truth data is needed to train the model, the RL agent chooses a suitable action to maximize the reward. Thus, the RL algorithm is actively tracking the state of the environment, and then adapts to any occurring changes by exploring a possible set of actions. In the presently disclosed method, RL is an attractive choice since many assumptions on the arrival and service rates can be dropped, and hence the provided framework is generic and can accommodate the time dependent arrival and service rates.

Given this representation, the RL agent selects an action to take and then accordingly the environment is transitioned into a new state. Based on the taken action, the agent receives a reward as a consequence of that action. The ultimate goal of an RL agent is to learn the best policy that maximizes its total expected reward. A value function is used to quantify how good or a bad is a certain policy, given a state-action pair. The optimal value function can be approximately computed through an iterative Q-learning. In the presently disclosed model-free reinforcement learning algorithm, the following update rule represented by Equation 10 is used to approximate the policy π that defines the optimal action at state s. In Equation 10, qₖ(s,a) quantifies the value of a given action, and γ is a discount factor that reduces the effect of future rewards on the current state, besides maintaining computation stability.

$${q}_{k} ( {S}_{k} , {A}_{k} ) ← {q}_{k} ( {S}_{k} , {A}_{k} ) + α [ {R}_{k + 1} + {γmax}_{{A}_{k + 1}} {q}_{k + 1} ( {S}_{k + 1} , {A}_{k + 1} ) - {q}_{k} ( {S}_{k} , {A}_{k} ) ] \qquad ( 10 )$$

Let d_{p,h}(k) indicate the selection of MF h by the RL agent to serve patient p for request k (or at times slot k). That is, d_{p,h}(k)=1 if the RL agent chooses MF h for patient p at time slot k, and 0 otherwise. Since the agent chooses only one MF to serve a patient request at any time slot, for any patient, the constraints represented by Equations 11 and 12 below hold for all patients and MFs.

$$\begin{matrix}{\sum }_{h = 1}^{H} {d}_{p , h} ( k ) = 1 , ∀ k , p & ( 11 ) \\ {d}_{p , h} ( k ) ∈ \{ 0 , 1 \} , ∀ p , h , k & ( 12 )\end{matrix}$$

To account for a patient's condition and better quality of service, a convex sum of two terms may be minimized: (i) the average of EST, and (ii) the STTP. Hence, the overall objective function can be written according to Equation 13 below. In Equation 13, [symbol] ₕ>>1 is a penalty for violating the STT constraint.

$${min}_{{d}_{p , h}} {lim}_{K -> ∞} [ \frac{1}{K} {\sum }_{p = 1}^{P} ( 1 - {ζ}_{p} ) {ω}_{p} [ {\sum }_{k = 0}^{K} {\sum }_{h = 1}^{H} {d}_{p , h} ( k ) {L}_{p , h} ( k ) ] + {\sum }_{p = 1}^{P} {ζ}_{p} {ω}_{pp} [ {\sum }_{k = 0}^{K} {\sum }_{h = 1}^{H} {ϑ}_{h} ℙ ( {L}_{p , h} ( k ) > {δ}_{p} ) ] ] \qquad ( 13 )$$

In order to optimize the performance and not violating the STTP constraint, ϑ>>1 is set to a very large value, i.e., the higher the penalty, the lower the STTP is. The above problem is an NP-hard non-convex multi-stage stochastic sequential decision optimization problem with integer constraints. A learning based RL algorithm may be used to solve it. To find the scheduling decisions, a learning-based policy may be considered where an RL agent interacts with an external environment (e.g., MFs and patients requests). For each request at time k, the agent observes some state sₖ and performs an action aₖ. Under aₖ, the state of the environment moves to a new state sₖ+1, and the agent receives a reward rₖ. The ultimate goal of learning is to maximize the expected cumulative discounted reward. For the patients scheduling problem, the components of a Markov decision process (MDP) process can be defined as follows. The reward is defined as the joint minimization of the EST and STTP metrics according to Equation 14 below.

$${r}_{p , k} = - ( 1 - {ζ}_{p} ) {ω}_{p} {\sum }_{h = 1}^{H} {d}_{p , h} ( k ) {L}_{p , h} ( k ) - {ζ}_{p} {ω}_{p} {\sum }_{h = 1}^{H} {ϑ}_{h} ℙ ( {L}_{p , h} ( k ) > {δ}_{o} ) ] \qquad ( 14 )$$

The state may be defined by the following three tuple: (1) the expected service time in the last k-steps, where k is an integer number, thus reflecting the rate of service at the MFs, (2) the number of patients in each MF, and (3) the average waiting time each MF. The action for every request is represented by a probability vector of the length H, whose h^{th} entry is equal to the probability of choosing the h^{th} MF to survey request t. The MF with the largest probability will be chosen.

Once a medical facility is selected, the patient is then assigned to the selected medical facility (block 210). For example, the patient assignment server 110 may transmit a scheduling request to a medical facility server 104 of the medical facility for a medical facility professional to confirm the scheduling request. In another example, the patient assignment server 110 may directly schedule the appointment for the patient at the selected medical facility for the appointment type in the scheduling request. The patient assignment server 110 may transmit a notification to the patient terminal 102 notifying the patient of the scheduled appointment.

The inventors have found that numerical results from computer Monte Carlo based simulations demonstrate a significant improvement of expected service time and service time tail probability metrics as compared to other competitive baselines. The inventors considered H=24 MFs equipped with different capabilities, where each facility has different equipment (i.e., number of clinical beds, emergency rooms, etc). To capture this heterogeneity among MFs, the inventors set the service rate μₕ=h/5 per minute, where h=1, H. In addition, the inventors set the shift parameter (fixed consultation time) to be equal to βₕ=2(1+h/H) , h. In addition, the consultation time for patient p, p, is assumed to follow a heavy-tailed Pareto distribution [20] as it is a commonly used distribution for such services, with shape factor of 2 and scale of 5 minutes, respectively. Unless otherwise stated, the inventors set P=1000, C=30 km/hr, δₚ=δ=20, and ζ=0.001. The inventors considered five groups of patients, each group has 200 patients. The arrival rates of each group are, respectively λₚ=0.001, 0.002, 0.01, 0.02, 0.03, where λ_{b} is the base arrival rate. Moreover, the inventors set the weight/criticality of each group to ωₚ=2, 4, 8, 3, 6, respectively. To initialize the provided method, one may begin by assuming uniform scheduling, q_{p,h}=1/H, τₕ=0.01, h.

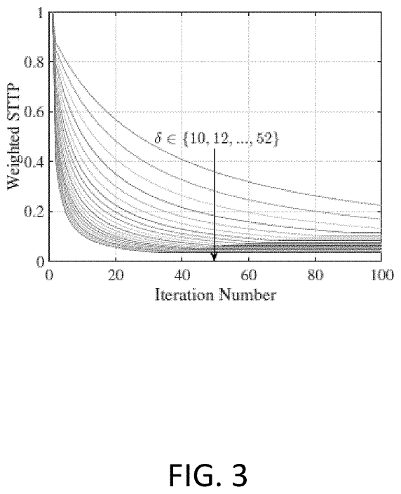

The model-based system performance is evaluated and compared with the state-of-the-other-algorithms and two competitive baselines. In particular, the inventors compared with Random Assignment (RA) (i.e., requests are assigned to medical facilities uniformly at random) and with a Proportional-service-rate Assignment (PA) policy. In this PA policy, the scheduling for patient requests are chosen to be proportional to the service rates of the MF, i.e., q_{p,h}=(β_{p,h}=1/μ_{p,h})/ₕ(β_{p,h}1/μ_{p,h}). FIG. 3 shows the convergence of the provided model-based method, where the weighted STTP is plotted for different values of δ, ranging from 10 to 52 minutes in an increment step of 2 minutes. It can be seen that the algorithm converges within 100 iterations which validates the efficiency of the provided algorithm.

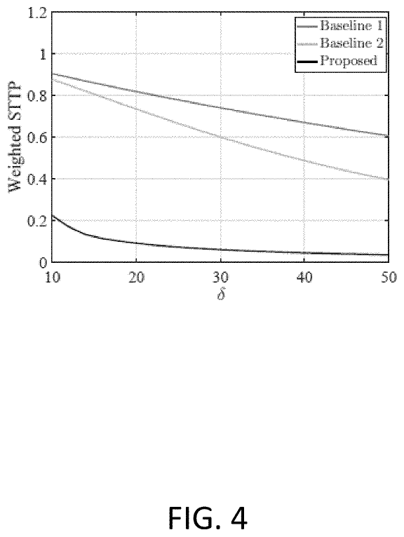

FIG. 4 depicts the performance of three optimization approaches. Two baseline solutions are compared with the joint τ and q optimization in the provided approach, where one optimizes τ only (Baseline 1), while the other only optimizes q (Baseline 2). The results highlights the importance of jointly optimizing τ and q over doing it one at a time on the weighted STTP. Moreover, the results show that optimizing τ is essential to the enhancement of the performance of the STTP metric.

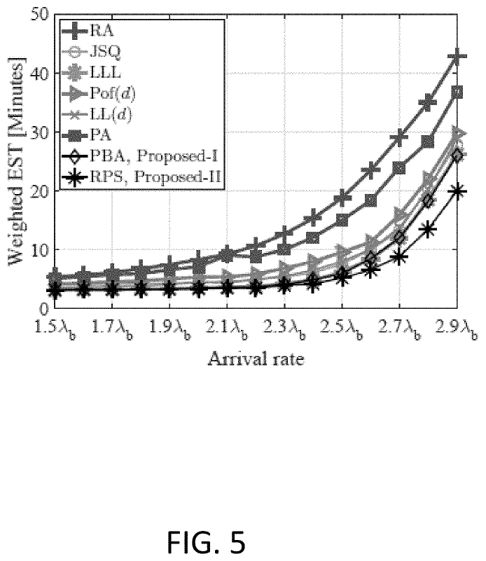

FIG. 5 shows the effect of increasing the rate of patients requests from 1.5λp to 2.9λp with an increment step of 0.1. In this figure, the provided online algorithms are compared with different online scheduling strategies. It was observed that the provided approach consistently performs the best among all considered approaches. In addition, at higher patient request rates, the provided approach still maintains low EST as compared to the most competitive baseline algorithm, i.e., least load left (LLL). For instance, at the arrival rate of 2.9λb, the provided strategy reduces the EST by around 25% compared to the LLL policy. Note that, unlike queue-length-based scheduling where only the queue length of patients counts, the provided approach differentiates among the different patients by prioritizing more the patients with higher weights/priority in order to offer them faster service and minimize the overall EST.

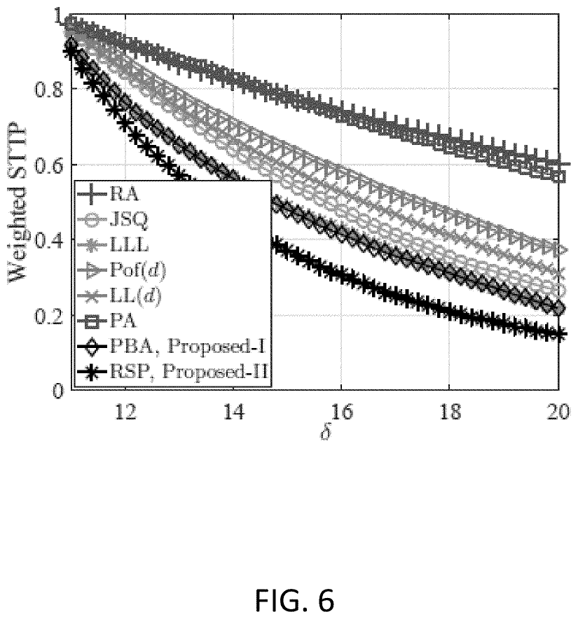

FIG. 6 shows the behavior of weighted STTP versus the threshold δₚ (in minutes). The provided approach finds the optimal weighted STTP by applying the alternating optimization algorithm. With optimized q, τ, the provided approach achieves the lowest STTP since it utilizes the resources better and accounts for both patient weights and MF capabilities. In the provided policy, higher-priority patients are prioritized, and thus their STTP is minimized, resulting in an overall decrease of the weighted STTP. In can be noted that this figure also represents the complementary cumulative distribution function (CCDF) of the aforementioned policies. For example, it was observed that the probability of the STTP being greater than 18 minutes is less than 20 percent for the provided policy, which is around 10% lower as compared to the LLL strategy.

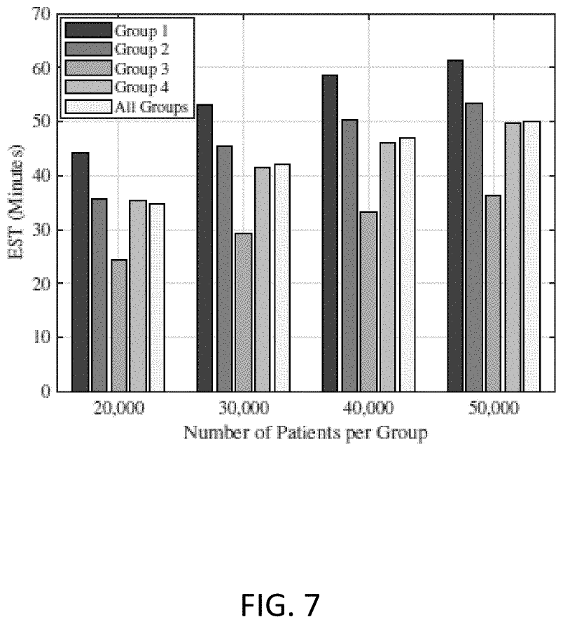

The model-free approach was also validated. The inventors constructed a simulator based on public data. The inventors used the provided statistics for arrival and service rates in to synthesize the dataset used in the experiments. FIG. 7 depicts the EST versus the number of patients per group. Four groups are identified based on the criticality of their health conditions, with Group 3 having patients with the highest priority/severity level, and Group 1 having the lowest. The EST increases as the number of patients per group increases, for all groups. The average EST is also depicted as a baseline. The results confirm that patients with the highest priority have the lowest EST (Group 3), while patients with lowest priority have the largest EST (Group 1). Moreover, STTP and EST results of the model-Free approach are shown in Table 1, where the performance gains of the provided approach are highlighted when compared with several benchmark results. The STTP and EST of the provided methods is much less than JSQ, its closest performing benchmark. Furthermore, the provided method's performance gain is much more significant when compared with the closest MF setup, which is a convenient choice for patients who wish to be medically examined. Another interesting benchmark that the provided method outperforms is the random selection scheme, which can be interpreted anthropomorphically as a varying preference of the patients. Again, the results highlight the importance of having a planned scheduling approach, such as the provided method, when examining the results of the random selection scheme.

TABLE 1

| Metric | STTP | EST (Minutes) |
|---|---|---|
| Provided | 0.016% | 19.28 |
| System | | |
| Random | 39.12% | 35.33 |
| Closest MF | 46.12% | 41.25 |
| PSP | 17.2% | 31.02 |
| JSQ | 9.71% | 27.11 |

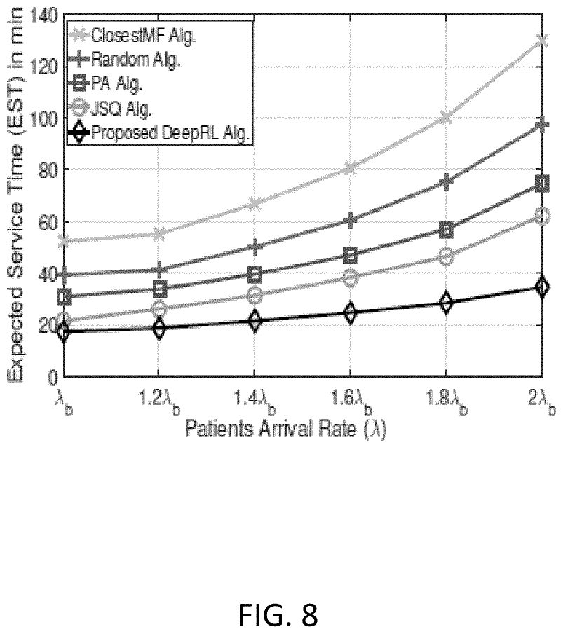

FIG. 8 depicts the performance of the DRL-based approach when increasing the rate of patients requests from λₚ to 2λₚ with an increment step of 0.2. In this figure, the provided approach is compared with different scheduling strategies. The provided DRL-approach consistently performs the best among all considered approaches, especially at higher patient request rates, and maintains low EST as compared to the most competitive JSQ baseline. Unlike queue-length-based scheduling where only the queue length of patients counts, the provided approach differentiates among the different patients by prioritizing the patients with higher weights/priority in order to offer them faster service and minimize the overall EST.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

The invention is claimed as follows:

1. A system for remote patient assignment comprising:
a memory; and
a processor in communication with the memory, the processor configured to:
receive a scheduling request from a computing device of a patient over a network;
receive information from each of a plurality of medical facilities;
determine an estimated service time for the patient to be treated at each of the plurality of medical facilities, wherein the estimated service time includes an estimated travel time for the patient to arrive at a respective medical facility, an estimated waiting time for the patient at the respective medical facility, and an estimated consultation time with a medical professional for the patient;
select a medical facility of the plurality of medical facilities that minimizes a sum of the determined estimated service time for the patient and a probability that the determined estimated service time for the patient is greater than or equal to a predefined threshold; and
assign the patient to the selected medical facility.

2. The system of claim 1, wherein the information from each of the plurality of medical facilities includes a current quantity of patients at each medical facility, an occupancy state of each medical facility, or an availability state of each medical facility.

3. The system of claim 1, wherein the estimated travel time for the patient to arrive at the respective medical facility is calculated according to the following equation:
$$𝔼 [ {t}_{p , h} ] = \frac{{D}_{p , h}}{C} + {∝}_{p , h}$$
wherein [symbol]
[t_{p,h}] is the estimated travel time for the patient,
p is the patient,
h is the respective medical facility,
t_{p,h} is a random travel time for the patient to reach the respective medical facility,
D_{p,h} is a distance between the patient and the respective medical facility,
C is a speed of travel, and
∝_{p,h} is a mean value of a random variable accounting for the randomness of the travel time.

4. The system of claim 1, wherein the estimated travel time for the patient to arrive at a respective medical facility is determined using at least one machine learning model including a neural network.

5. The system of claim 1, wherein the estimated waiting time for the patient at the respective medical facility is calculated based on a total quantity of patients at the respective medical facility, a quantity of patients waiting to be seen at the respective medical facility, and a condition of the patient.

6. The system of claim 1, wherein the estimated waiting time for the patient at the respective medical facility is calculated based on each patient being served on a first-come first-served basis at the respective medical facility.

7. The system of claim 1, wherein the estimated waiting time for the patient at the respective medical facility is an average waiting time at the respective medical facility.

8. The system of claim 1, wherein the estimated consultation time with a medical professional for the patient is determined by a shifted exponential distribution function.

9. The system of claim 8, wherein the shifted exponential distribution function is defined by:
$${f}_{p , h} ( s ) = \{ \begin{matrix}{μ}_{p , h} {e}^{- {μ}_{p , h} ( s - {β}_{p , h} )} & s \geq {β}_{p , h} \\ 0 & s < {β}_{p , h}\end{matrix}$$
wherein
$${μ}_{p , h} = {μ}_{h} / {V}_{p} , \\ {β}_{p , h} = {β}_{h} {V}_{p} ,$$
p is the patient,
h is the respective medical facility,
βₕ is a minimum consultation time at the respective medical facility,
μₕ is an average consultation time at the respective medical facility,
Vₚ is a total time of a visit by a patient at the respective medical facility, and
s is a type of service for the consultation.

10. The system of claim 1, wherein the information from each of the plurality of medical facilities is updated at regular intervals.

11. The system of claim 1, wherein the medical facility of the plurality of medical facilities is selected according to the below relationship:
$${min}_{q , τ} {\sum }_{p = 1}^{P} {ω}_{p} [ ( 1 - {ζ}_{p} ) 𝔼 [ {L}_{p} ] + {ζ}_{p} ℙ ( {L}_{p} \geq {δ}_{p} ) ]$$
wherein
ωₚ is a level of severity of a health condition of the patient, [symbol]
ₚ is a trade-off factor, for the patient, that determines the relative significance of STTP,
Lₚ is a service time of the patient,
δₚ is the predefined threshold, and [symbol] (Lₚ≥δₚ) is the probability that the determined estimated service time for the patient is greater than or equal to the predefined threshold.

12. The system of claim 1, wherein the medical facility of the plurality of medical facilities is selected based on reinforcement learning.

13. The system of claim 12, wherein a reward of the reinforcement learning is defined as the joint minimization of the estimated service time and the probability that the determined estimated service time for the patient is greater than or equal to a predefined threshold,
wherein a state of the reinforcement learning is defined by: (i) an expected service time in the last k-steps, wherein k is an integer, (ii) a quantity of patients in each medical facility, and (iii) an average waiting time in each medical facility, and
wherein an action of the reinforcement learning is represented by a probability vector of choosing a medical facility to serve the scheduling request.

14. A method for remotely assigning a patient to a medical facility comprising
receiving a scheduling request from a computing device of a patient over a network;
receiving information from each of a plurality of medical facilities;
determining an estimated service time for the patient to be treated at each of the plurality of medical facilities, wherein the estimated service time includes an estimated travel time for the patient to arrive at a respective medical facility, an estimated waiting time for the patient at the respective medical facility, and an estimated consultation time with a medical professional for the patient;
selecting a medical facility of the plurality of medical facilities that minimizes a sum of the determined estimated service time for the patient and a probability that the determined estimated service time for the patient is greater than or equal to a predefined threshold; and
assigning the patient to the selected medical facility.

15. The method of claim 14, wherein the estimated service time for the patient to be treated at each of the plurality of medical facilities is calculated according to:
$$𝔼 [ {L}_{p} ] = {\sum }_{h = 1}^{H} {q}_{p , h} [ ( \frac{{D}_{p , h}}{C} + {∝}_{p , h} ) + \frac{{Λ}_{h} 𝔼 [ {S}_{h}^{2} ]}{2 ( 1 - {Λ}_{h} 𝔼 [ {S}_{h} ] )} + 𝔼 [ {S}_{p , h} ] ]$$
wherein
$$𝔼 [ {S}_{h} ] = {\sum }_{p = 1}^{P} \frac{{q}_{p , h} {λ}_{p}}{{Λ}_{h}} ( 𝔼 [ {S}_{p , h} ] ) , \\ 𝔼 [ {S}_{p , h} ] = {β}_{p , h} + \frac{1}{{μ}_{p , h}} ,$$
Λₕ is an arrival rate of requests at the respective medical facility,
Lₚ is a service time of the patient,
$${μ}_{p , h} = {μ}_{h} / {V}_{p} , \\ {β}_{p , h} = {β}_{h} {V}_{p} ,$$
p is the patient,
h is the respective medical facility,
βₕ is a minimum consultation time at the respective medical facility,
μₕ is an average consultation time at the respective medical facility,
Vₚ is a total time of a visit by a patient at the respective medical facility,
q_{p,h} is a probability of serving a request of a patient from the respective medical facility,
D_{p,h} is a distance between the patient and the respective medical facility,
C is a speed of travel, and
∝_{p,h} is a mean value of a random variable accounting for the randomness of the travel time.

16. The method of claim 14, wherein the probability that the determined estimated service time for the patient is greater than or equal to the predefined threshold is determined according to the below relationship:
$$ℙ ( {L}_{p} \geq {δ}_{p} ) \leq {\sum }_{h = 1}^{H} \frac{{q}_{p , h} ( 1 - {ρ}_{h} ) {τ}_{h} {e}^{- {τ}_{h} {δ}_{p}}}{{τ}_{h} - {Λ}_{h} ( {M}_{h} ( {τ}_{h} ) - 1} \times ( {e}^{{D}_{p , h} {τ}_{h} / C} {e}^{{σ}_{p , h}^{2} {τ}_{p , h}^{2} {τ}_{h}^{2} / 2} ) ( \frac{{μ}_{p , h} {e}^{{β}_{p , h} {τ}_{h}}}{{α}_{p , h} - {τ}_{h}} )$$
wherein
$$0 < {τ}_{h} < {μ}_{p , h} , \\ {ρ}_{h} = {Λ}_{h} 𝔼 [ {S}_{h} ] , \\ 𝔼 [ {S}_{h} ] = {\sum }_{p = 1}^{P} \frac{{q}_{p , h} {λ}_{p}}{{Λ}_{h}} ( 𝔼 [ {S}_{p , h} ] ) , \\ 𝔼 [ {S}_{p , h} ] = {β}_{p , h} + \frac{1}{{μ}_{p , h}} ,$$
Lₚ is a service time of the patient,
Λₕ is an arrival rate of requests at the respective medical facility,
$${μ}_{p , h} = {μ}_{h} / {V}_{p} , \\ {β}_{p , h} = {β}_{h} {V}_{p} ,$$
p is the patient,
h is the respective medical facility,
βₕ is a minimum consultation time at the respective medical facility,
μₕ is an average consultation time at the respective medical facility,
Vₚ is a total time of a visit by a patient at the respective medical facility,
q_{p,h} is a probability of serving a request of a patient from the respective medical facility,
δₚ is the predefined threshold.

17. The method of claim 14, wherein selecting the medical facility of the plurality of medical facilities includes minimizing the weighted sum of the determined estimated service time for the patient while the probability that the determined estimated service time for the patient is greater than or equal to a predefined threshold is fixed, and minimizing the probability that the determined estimated service time for the patient is greater than or equal to a predefined threshold is fixed while the weighted sum of the determined estimated service time for the patient is fixed.

18. A non-transitory, computer-readable medium storing instructions, which when executed by a processor, cause the processor to:
receive a scheduling request from a computing device of a patient over a network;
receive information from each of a plurality of medical facilities;
determine an estimated service time for the patient to be treated at each of the plurality of medical facilities, wherein the estimated service time includes an estimated travel time for the patient to arrive at a respective medical facility, an estimated waiting time for the patient at the respective medical facility, and an estimated consultation time with a medical professional for the patient;
select a medical facility of the plurality of medical facilities that minimizes a sum of the determined estimated service time for the patient and a probability that the determined estimated service time for the patient is greater than or equal to a predefined threshold; and
assign the patient to the selected medical facility.

19. The non-transitory, computer-readable medium of claim 18, wherein the medical facility of the plurality of medical facilities is selected based on randomized prioritized scheduling.

20. The non-transitory, computer-readable medium of claim 18, wherein the medical facility of the plurality of medical facilities is selected based on a pull-based algorithm.
